(19) United States
(12) Patent Application Publication
(10) Pub. No.: US 2025/0139108 A1
(43) Pub. Date: May 1, 2025

(54) Design Strategies to Prevent Data Hallucination in Large Language Models for the Medical Field

(71) Applicant: Miao Cui, Flushing, NY (US)

(72) Inventor: Miao Cui, Flushing, NY (US)

(21) Appl. No.: 19/004,302

(22) Filed: Dec. 28, 2024

(51) Int. Cl. G06F 16/2458; G16H 50/70

(52) U.S. Cl. CPC G06F 16/2471; G16H 50/70

(57) ABSTRACT

This invention proposes novel prompt engineering strategies to ensure the accuracy of large language models in medical applications, effectively mitigating the risk of data hallucination. The methods include stepwise database search designs, reference-providing mechanisms to enhance operational transparency and facilitate manual verification, and the integration of multilingual support schemes. These innovative prompt engineering designs significantly improve the reliability, transparency, and clinical applicability of information generated by natural language models.

### BACKGROUND OF THE INVENTION

Large language models (LLMs), such as GPT, have revolutionized the field of artificial intelligence, especially in natural language processing. Nevertheless, current LLMs often generate responses that are inaccurate, unreliable, or entirely fabricated, a phenomenon commonly referred to as “data hallucination.” Moreover, these models fail to reveal their retrieval processes and the mechanisms underlying their generated responses. Consequently, users cannot directly ascertain the sources or materials upon which the model's conclusions are based, making it difficult to evaluate the accuracy and reliability of its responses. Although language models exhibit strong comprehension of English in specialized domains, their understanding of non-English languages is inconsistent. This inconsistency represents another significant cause of frequent “data hallucination” in professional contexts. These critical shortcomings render language models unsuitable for broad adoption in specialized fields such as clinical medicine, where accuracy and reliability are paramount.

To address these challenges, this invention proposes a novel prompt engineering strategy aimed at effectively mitigating data hallucination during the application of language models in clinical medicine. Additionally, it seeks to enhance the transparency of model operations and provide robust multilingual support.

### SUMMARY OF THE INVENTION

This invention presents an innovative approach to prompt engineering for natural language models within the medical domain, encompassing the following advancements: 1. Employing a structured search design to systematically access trustworthy medical data sources, thereby improving the accuracy of the language model's generated responses. 2. Incorporating a mechanism to list references used in response generation, transitioning the model's operational framework from a “black box” to a supervised system. This design enables users to manually verify information sources as needed, ensuring data reliability. 3. Developing a multilingual support framework to maintain accuracy during non-English usage scenarios.

### DETAILED DESCRIPTION OF THE INVENTION

1. Step-Wise Search Design to Mitigate Data Hallucination: When users query medical concepts, the language model employs a systematic, step-wise search strategy: 1) Initial Search: The model initially searches for relevant information within internally trained, authoritative medical guidelines (e.g., WHO Classification of Tumours, AJCC Cancer Staging System). 2) External Database Search: If the query cannot be fully resolved, the model proceeds to search the following external databases in order of relevance: a) Authoritative medical journal databases (e.g., PubMed): Conducts in-depth searches using specific medical keywords. b) Other open libraries and academic databases: Broadens the search to include a wider range of academic resources. c) Google: Uses general search engines to retrieve supplementary information

The prompt engineering design: ‘When explaining a concept, please first search for the concept in your pre-trained database and respond using simple, beginner-friendly language with a rigorous writing style based on your pre-trained databases. Use vivid examples for explanation. All responses should be fact-based: Set the operating temperature to 0, and prioritize factual, accurate, and unbiased information in all outputs. When the inquiry is not covered by the pre-trained database, perform the following searches in sequence using the most appropriate keywords related to the specific topic and display all results: 1. First, search through actions via eutils.ncbi.nlm.nih.gov; 2. Then, proceed with actions to search other databases; 3. Finally, conduct searches through Google and Google Books.’

2. Supervising Model Operations for Transparent Information Retrieval To enable supervised information retrieval, the model implements the following strategies: Each piece of information retrieved from external databases or Google is cited in parentheses immediately following the content, with embedded clickable links. A comprehensive bibliography of references used to generate the response is appended at the end. This practice ensures transparency and enables users to directly verify the provided information.

The prompt engineering design: ‘When mentioning external database searches, please insert APA-style in-text citations, including the article title, authors, journal name, publication date, PMID number throughout the reply, and provide a comprehensive bibliography with clickable URLs at the end. Use only the API for references; do not fabricate any references. When conducting online searches and gathering information, include specific source links in parentheses immediately following the content you have retrieved for easy verification. If the search content contains an image, display the image directly in the search results and add a link to the corresponding page for the image’

3. Multilingual Support: This invention improves the accuracy of multilingual interactions through the following measures: a) Non-English queries are first translated into English to enhance the model's understanding of the user's inquiry. b) The English translation is processed to generate accurate results. c) The resulting output is then translated back into the user's original language for easier comprehension. This internally designed dual-translation process mitigates the risk of “data hallucination” during understanding and reasoning, ensuring the delivery of high-quality responses.

The prompt engineering design: ‘When a query is made in a language other than English, please first automatically translate the query into English before executing the request, and generate the results in the same language as the query in the end.’

1. A proposed framework to mitigate the risk of data hallucination in large language models when processing medical information, thereby ensuring response accuracy: 1. Stepwise search design: The model performs sequential searches through internal training data, PubMed, other open-access libraries and academic databases, and the Google search engine, prioritizing query relevance. 2. Transparency in sources and citations: For external database queries or Google searches, the model offers openly verifiable source links and citations to facilitate manual source validation by users. 3. Multilingual handling framework: A dedicated framework is incorporated to enable the effective processing of multilingual content by the large language model.
